(19) United States
(12) Patent Application Publication
(10) Pub. No.: US 2023/0080017 A1
(43) Pub. Date: Mar. 16, 2023

(54) COATED MICROCAPSULES AND METHODS FOR THE PRODUCTION THEREOF

(71) Applicant: ZANTEBIO LIMITED, Dublin (IE)

(72) Inventors: Sinead BLEIEL, Dublin (IE); Robert KENT, Cork (IE); Maja SEVERIC, Dublin (IE)

(73) Assignee: ZANTEBIO LIMITED, Dublin (IE)

(21) Appl. No.: 17/801,079

(22) PCT Filed: Feb. 19, 2019
(86) PCT No.: PCT/EP2021/054238

§ 371 Date: Aug. 19, 2022

(30) Foreign Application Priority Data
Feb. 19, 2020 (GB) 2002323.0

(51) Int. Cl. A61K 9/50; A61K 35/741; A23P 20/10; A23P 10/35; A23P 30/25; A23L 33/18; A23L 33/135

(52) U.S. Cl. CPC A61K 9/5015; A61K 35/741; A61K 9/5089; A23P 20/11; A23P 10/35; A23P 30/25; A23L 33/18; A23L 33/135; A61K 2035/115

(57) ABSTRACT

A method of producing coated microcapsules comprises the steps of producing microcapsules by cold gelation having a denatured or hydrolysed protein matrix and an active agent contained within the matrix, and drying the microcapsules. A meltable coating composition comprising wax and oil and configured to have a melting point of about 70° C. to about 100° C. is heated to a temperature above the melting point of the meltable coating composition to melt the meltable coating composition, and the microcapsules are coated with the melted meltable coating composition

### FIELD OF THE INVENTION

The present invention relates to coated microcapsules and methods for the production thereof. The invention also relates to food and beverage products containing coated microcapsules.

### BACKGROUND TO THE INVENTION

Methods of making microcapsules using milk and vegetable proteins are described in the literature. Generally, the microcapsules have a micron-sized dimension (i.e. an average dimension of 50-200 microns, and comprise a crosslinked (denatured or hydrolysed) protein matrix containing an active agent contained and protected within the matrix. The microcapsules may have a mononuclear morphology, where the active agent is provided by a single core contained within a protein shell, or may be multinuclear in which discrete pockets of active agent are homogenously distributed throughout a protein matrix. Generally, the protein matrix of the microcapsules is gastric resistant and susceptible for break-up in the ileum, thus enabling delivery of active agents through the acidic stomach conditions intact for release in the proximal ileum. Examples of microcapsule technology is described in the following documents:

WO2009/119041 describes milk protein microcapsules formed from whey protein and used to deliver probiotic bacteria through the stomach and into the ileum. The microcapsules are produced by heat denaturing whey protein, mixing a suspension of the denatured whey protein and probiotic bacteria, and extruding the mixture into microdroplets using a vibrating nozzle. The microdroplets are immediately immersed in an acidic gelation bath which crosslinks the denatured protein to form gastric resistant and ileal sensitive microcapsules.

WO2014/198787 describes milk protein microcapsules for delivering creatine to the ileum. The microcapsules are formed in a manner similar to that described in WO2019/119041, except that the protein is hydrolysed, the microdroplets include a liquid ester, and the gelation bath comprises a basic solution that converts the liquid ester into a salt, which in turn polymerises the hydrolysed protein to create the gastric resistant and ileal sensitive microcapsules.

WO2016/096929 described pea protein microcapsules, and methods of preparing high concentration, soluble, pea protein useful for making microcapsules. The invention describes the use of a concentric nozzle extruder for co-extrusion of a protein suspension (matrix) and a suspension of active agent forming core-shell microdroplets which are cured in a gelation bath forming core-shell microcapsules.

Generally, microcapsules are sieved after gelation, and then dried using for example a fluidised bed drying technology to a moisture content of less than 5%. The resultant microcapsule powder displays free-flowing powder characteristics and absence of agglomerated material. Typically, these microcapsules contain bioactive functional ingredients such as probiotic bacteria, omega oils or vitamin/mineral mixes. The drying process does not have a significant deleterious effect on the functionality of these ingredients.

Post drying these microcapsules are resistant to moisture uptake and remain insoluble in aqueous conditions. Although the microcapsules remain insoluble, the external layer of the material can soften and take up an amount of water in aqueous or humid conditions. The water activity of the microcapsules in the post dried state can also increase in high humidity environments. This is undesirable as the moisture can have deleterious effects on the active agent contained within the microcapsules.

Microparticles having wax or oil coatings as moisture barriers are described in the literature, see for example WO2006/066389, WO2010/111347 and US20080026108. While oil or wax coatings do improve stability of the microparticles, a problem with oil coated microparticles is that they are not suited to many applications such as foods or beverages that require a heat-treatment, as the heat employed melts the coating and renders the microparticles unprotected. This is especially problematical for microparticles made using the methods described in the prior art where the uncoated microparticles are especially heat labile as they are unprotected.

It is an object of the invention to overcome at least one of the above-referenced problems.

### SUMMARY OF THE INVENTION

The Applicant has addressed the problem of the prior art by providing an active agent encapsulated in a cold-gelated microcapsule where the matrix of the microcapsule comprises polymerised denatured or hydrolysed protein (which provides protection to the active agent when it is exposed to heat) and coating the microcapsule with a meltable coating composition that is configured to melt at a temperature above the temperature of many commercial heat treatments such as pasteurisation. A coating composition with a melting temperature of 70-100° C. is suitable, as such a coating composition will remain intact as a protecting layer when the coated microcapsules are heat treated by e.g. pasteurisation, and yet melts and becomes processable at a temperature which is generally not damaging to the active agent that is encapsulated. The applicant has discovered that in the context of coating cold-gelated microcapsules, the use of a mixture of wax and oil and waxes, oils and/or protein, provides a coating composition that is processable and provides suitable melting temperatures to meet the above requirements. The Applicant has also discovered that mixing a wax (optionally a combination of waxes) with an oil having a different melting point to the wax, provides a coating material that easy to handle, has improved dispersibility, and provides a very homogenous coating on protein microcapsules. The higher melting point component builds a more robust micro-bead structure and enhances microbead and barrier strength and tensile properties. The lower melting point component increases the adherence of the coating to the surface to enable the generation of a hydrophobic layer on the protein microcapsule surface. Coatings made of carnuba wax and coconut oil, carnuba wax and beeswax, and carnuba wax, beeswax and coconut oil, have been found to be particularly effective as moisture barriers for cold gelated microcapsules (FIGS. 5 and 6).

Broadly, the invention provides a method of producing coated microcapsules comprising the steps of:
producing microcapsules having a protein matrix and an active agent contained within the matrix;
drying the microcapsules;
providing a meltable coating composition comprising wax (and ideally oil) that is solid at room temperature; and
coating the microcapsules with the coating composition.

Typically, the meltable coating composition employed in the method and products of the invention has a melting point above room temperature, for example at least 25 ° C. , 30° C., 40° C., 50° C., 60° C., 70° C., 80° C., or 90° C. This allows the coating composition to be heated to a processing temperature where it can flow and is sufficiently dispersible to coat the microcapsules. Typically, the coating composition employed in the method and products of the invention has a melting point of less than 100° C., 95° C. or 90° C. This ensures that the coating composition does not heat damage the microcapsules and in particular the active agent. Some active agents, for example probiotic bacteria, and susceptible to heat damage.

The method generally comprises heating the meltable coating composition to a temperature above the melting point of the coating composition, and then coating the microcapsules with the heated coating composition. The coating composition may be obtained by heating the wax and oil components to a melting temperature, and then homogenising the heated components to provide the coating composition.

In one embodiment, the protein is denatured or hydrolysed protein.

In one embodiment, the protein is milk or vegetable protein.

In one embodiment, the microcapsules are produced by produced by extrusion of microdroplets, and immersion of the microdroplets in a gelation bath.

In one embodiment, the coating composition comprises 50-90% wax and 10-50% oil.

In one embodiment, the coating composition comprises 60-70% wax and 25-40% oil.

In one embodiment, the wax is selected from beeswax, carnuba wax, or a combination thereof.

In one embodiment, the wax comprises carnauba wax.

In one embodiment, the wax comprises beeswax and carnauba wax.

In one embodiment, the oil is selected from coconut oil, palm oil, sunflower oil and cocoa butter (any of which may be hydrogenated).

In one embodiment, the coating composition comprises carnauba wax, beeswax, and an oil (typically coconut oil). In one embodiment, the coating composition comprises carnauba wax, beeswax, coconut oil and one or more of palm oil (optionally hydrogenated), sunflower oil (optionally hydrogenated) and cocoa butter.

In one embodiment, the coating composition comprises 30-35% carnauba wax, 30-35% beeswax, and 30-35% coconut oil.

In one embodiment, the drying step comprises drying the microcapsules to water activity Aw of less than 0.3 at 25° C.

In one embodiment, the drying step comprises drying the microcapsules to water activity Aw of less than 0.2 at 25° C.

In one embodiment, the drying step comprises a primary drying stage and a secondary drying stage in a low humidity environment.

In one embodiment, the coating step comprises mixing the heated coating composition and dried microcapsules in a coating chamber until the coating composition has coated the microcapsules and solidified.

In one embodiment, the coating step comprises spraying the heated coating comprising on to the dried microcapsules on a fluidised bed.

In one embodiment, the heated coating composition is homogenised at high shear prior to coating the microcapsules.

In one embodiment, the coating step employs about 40-60% coating composition and about 40-60% dried microcapsules.

In one embodiment, the matrix comprises denatured vegetable or milk protein.

In one embodiment, the active agent comprises a probiotic bacteria, oil, or micronutrient composition.

In one embodiment, the microcapsules are produced by provided extruded microdroplets comprising denatured or hydrolysed protein and an active agent into a gelation bath, and gelation of the microdroplets in the gelation bath to provide microcapsules.

In one embodiment, the microcapsules are multinuclear, in which the microcapsules are typically produced by providing a suspension comprising denatured or hydrolysed protein matrix and an active agent, and extruding microdroplets of the suspension into a gelation bath.

In one embodiment, the microcapsules are mononuclear, in which the microcapsules are typically produced by providing a first liquid composition comprising denatured or hydrolysed protein matrix and a second liquid composition comprising an active agent, and co-extruding the first and second liquid compositions using concentric nozzles in which the first liquid composition is extruded through an outer nozzle and the second liquid composition is extruded through an inner nozzle to form core-shell microdroplets, and gelation of the core-shell microdroplets in a gelation bath.

In any embodiment, the microcapsules are produced by providing a first liquid composition comprising denatured or hydrolysed protein matrix and an active agent, and co-extruding the first liquid compositions and the meltable coating composition using concentric nozzles in which the first liquid composition is extruded through an inner nozzle and the meltable coating composition is extruded through an outer nozzle to form core-shell microdroplets in which the shell comprises the meltable coating composition, and gelation of the core-shell microdroplets in a gelation bath. The Applicant has discovered that coated microcapsules formed using this method require less coating composition, for example a weight ratio of first composition to coating composition of 1:10 to 5:10.

The invention also provides a microcapsule comprising:
a crosslinked protein matrix;
an active agent contained within the matrix;
wherein the microcapsule is coating with a solidified coating composition comprising wax
and oil and/or protein.
and having a melting point above 25° C.

In one embodiment, the coating composition comprises 50-90% wax and 10-50% oil

In one embodiment, the coating composition comprises 60-70% wax and 30-40% oil.

In one embodiment, the coating composition comprises protein, typically denatured or hydrolysed protein.

In one embodiment, the coating composition comprises=40-60% protein, 30-40% wax and 10-20% oil

In one embodiment, the wax comprises beeswax.

In one embodiment, the wax comprises carnauba wax.

In one embodiment, the wax comprises beeswax and carnauba wax.

In one embodiment, the oil comprises coconut oil.

In one embodiment, the coating composition comprises carnauba wax, beeswax, and coconut oil.

In one embodiment, the coating composition comprises 30-35% carnauba wax, 30-35% beeswax, and 30-35% coconut oil.

In one embodiment, the microcapsule has a water activity Aw of less than 0.3 at 25° C.

In one embodiment, the microcapsule has a water activity Aw of less than 0.2 at 25° C.

In one embodiment, the microcapsule is a mononuclear microcapsule.

In one embodiment, the microcapsule is a multinuclear microcapsule.

In one embodiment, the protein is selected from a milk or vegetable protein, which is denatured.

In one embodiment, the invention provides a microcapsule comprising:
a crosslinked denatured vegetable or milk protein matrix;
an active agent contained within the matrix selected from an oil, a bacterial composition, or a micronutrient;
wherein the microcapsule is coating with a meltable coating composition comprising wax and oil configured to be solid at ambient temperature.

In one embodiment, the invention provides a microcapsule comprising:
a crosslinked denatured vegetable or milk protein matrix;
an active agent contained within the matrix, in which the active agent is a probiotic bacteria preparation;
wherein the microcapsule is coating with a meltable coating composition comprising beeswax and oil configured to be solid at ambient temperature.

In one embodiment, the invention provides a microcapsule comprising:
a crosslinked denatured vegetable or milk protein matrix;
an active agent contained within the matrix;
wherein the microcapsule is coating with a meltable coating composition comprising beeswax, carnauba wax and oil configured to be solid at ambient temperature.

In any embodiment, the coating composition is configured to have a melting point from 25-100° C., 50-100° C., 60-100° C., 70-100° C., 80-100° C., 90-100° C., or about 95° C.

The invention also provides a comestible (i.e. edible) composition comprising microcapsules to the invention. The comestible composition may be a food, beverage, or nutritional supplement. In one embodiment, the comestible composition is selected from a beverage and a gum. The comestible composition may comprise 0.1 to 10% coated microcapsules of the invention.

In another aspect, the invention provides a heat-treated composition comprising microcapsules according to the invention, in which the composition is typically heat treated at a temperature less than the melting point of the coating composition.

In any embodiment, the composition is pasteurised.

In another aspect, the invention provides a method of making a heat-treated composition comprising the steps of providing a composition comprising microcapsules according to the invention, and heat treating the composition at a temperature preferably less than the melting point of the coating composition.

In any embodiment, the heat treatment is pasteurisation.

In any embodiment, the active agent comprises probiotic bacteria.

In any embodiment, the composition is a food, beverage or nutritional supplement.

The Applicant has also discovered that core-shell microcapsules formed by cold gelation in which both the core and shell comprise polymerised denatured or hydrolysed protein are very heat stable, and can be heat treated to commercial sterilisation temperatures without significant loss of viability of the encapsuled active agent. This has been tested with a probiotic as active agent and UHT heat treatment with the result that the loss of viability of probiotic bacteria in the microcapsules was very low. This allows probiotic bacteria to be provided in shelf-stable compositions, for example shelf-stable beverages.

In another aspect, the invention provides a core-shell microcapsule, in which the core comprises an active agent contained within a polymerised protein matrix and in which the shell comprises polymerised protein.

In any embodiment, the protein is milk or plant protein, for example whey protein, casein, soy, zein, rice or pea protein.

In any embodiment, the protein in the shell and core is denatured or hydrolysed.

In any embodiment, the microcapsule is produced by cold-gelation.

In any embodiment, the microcapsule is produced with double concentric nozzle.

In any embodiment, the active agent is heat labile, for example a cell such as a probiotic bacterium or a heat-labile micronutrient or drug such as vitamin D, creatine or a biological drug.

The invention also provides a composition comprising a multiplicity of core-shell microcapsules according to the invention.

In any embodiment, the composition is heat-sterilised.

In any embodiment, the active agent is a probiotic bacterium.

In any embodiment, the composition is a shelf-stable beverage.

In another aspect, the invention provides a method of making core-shell microcapsules comprising the steps of:
providing a first liquid composition comprising denatured or hydrolysed protein matrix and an active agent;
providing a coating composition comprising or consisting essentially of denatured or hydrolysed protein;
co-extruding the first liquid compositions and the coating composition using concentric nozzles in which the first liquid composition is extruded through an inner nozzle and the coating composition is extruded through an outer nozzle to form core-shell microdroplets in which the shell comprises the coating composition; and gelation of the core-shell microdroplets in a gelation bath.

In any embodiment, the coating composition comprises wax, oil or a mixture of wax and oil.

In any embodiment, the protein is denatured protein. In any embodiment, the gelling bath is configured to polymerise the protein of the core and the shell.

The invention also provides core-shell microcapsules obtainable according to the invention.

The invention also provides a method of making a heat-treated composition (for example a food, beverage or nutritional supplement) comprising the steps of:

making core-shell microparticles according to the invention; adding the core-shell microparticles to additional components to make a composition; and heat treating the composition.

The heat treatment may be a sterilisation treatment, for example a Ultra High Temperature (UHT) treatment (e.g. 143° C.×3 second). Sterilisation generally means heating the composition to at least 100° C.

In any embodiment, the denatured protein comprises whey-containing dairy protein or vegetable protein.

In any embodiment, the denatured protein comprises whey protein isolate, whey protein concentrate, milk protein concentrate, or pea protein isolate.

In any embodiment, the matrix (or shell) is formed from a denatured protein solution having a protein concentration of 1-12%, 1-10%, 1-5%, 1-3%, 4-12%, 4-8%, 4-12% (w/v). When the protein is pea protein, the protein concentration is suitably 7-9%, preferably about 8% (w/v). When the protein is whey protein, the protein concentration is suitably 10-12%, preferably about 11% (w/v). When the protein is milk protein, the protein concentration is suitably 4-6%, preferably about 5% (w/v).

In any embodiment, the denatured protein solution is prepared by heat denaturation at a temperature of 70-90° C. for a period of 30-60 minutes. Preferably, the denatured protein solution is fully or at least 90% denatured.

In any embodiment, the denatured protein solution is rapidly cooled immediately after heat denaturation to prevent immediate gelation of the solution.

Other aspects and preferred embodiments of the invention are defined and described in the other claims set out below.

### BRIEF DESCRIPTION OF THE FIGURES

FIG. 1 . Images dry oil/wax coated microparticles. Image A shows microparticles after production (×100 magnification). Image B shows microparticles in water after 48 hours (×100 magnification). No difference recognised in surface topography after dispersion/immersion in water after 48 hour; and not water absorption recognised.

FIG. 2 . Images A and B show cold gelated microparticles before oil/wax coating application. Image B shows micro-capsules after oil+wax coating application. Images C and D show cold gelated microbeads coated with oil and wax in different proportions but not visual difference and no size difference.

FIG. 3. Image A confirms the presence of the live probiotic bacteria load in dry oil/wax coated microparticles. Cell counts can range from 1 billion to 100 billion CFU per gram and the process is sustained reproduction capacity from batch to batch. FIG. 3B shows dried oil/wax coated microparticles after high humidity storage 75% (A) and after fortification in a UHT beverage for 3 months (C). Microscopy shows no change in surface topography after stress exposure to humidity or UHT beverage environments.

FIG. 4. Microscopy images to verify the natural human digestion of coated micro-capsules during in vitro human digestion. Image 4A represented digesta after stomach incubation step. Image 4B illustrates digestion after 15 minutes intestinal incubation. Image 4C illustrates break down after 30 minute intestinal digestion and image 4D illustrates full break down after 45 minute intestinal digestion.

FIG. 5. Humidity measurements of control and coated microcapsules after 7.5 days at 75% high humidity. Dashed line represents control (porous microbeads) and continuous line represents coated microbeads that resist humidity and moisture update.

NOTE: The gain in weight while storing the samples at high relative humidity atmosphere is linked with the affinity with water. Hence, a low water gain in the samples during exposure to high humidity will correspond with those samples with highest hydrophobic profile i.e. microcapsules with a coating of oil and/or wax.

FIG. 6A. Humidity measurements of control (porous) microbeads and various coated microcapsules after storage at 75% high humidity. Different percent coatings and different ratios of oil-wax coating will generate different levels of resistance to high humidity environments.

FIG. 6B shows the comparison of weight gain vs. moisture uptake values for control microbeads and various other wax coated microcapsules after exposure to 75% high humidity. Different percent coatings and different ratios of oil-wax will generate different levels of resistance to high humidity environments.

FIG. 6C illustrates the difference humidity resistance between the control (porous)beads (high level single blue line) after 33 days at 75% high humidity using various coating combinations and coating ingredient ratios (all other coloured lines). A significant increase in moisture resistance is evident after 33 days when a coating layer is applied.

FIG. 6D compare the weight gain and moisture for various coated microparticles relative the control porous microbeads after 3.3 days storage at 75% relative humidity.

FIG. 7. Explanation for coating principle. NOTE: Generation of hydrophobic coating on cold gelated microparticles or microbeads, should have a small contact angle to ensure even coverage and continuous coating with no porous formation on the surface of the final product; and a high contact angle of water on the surface of the coating, to assure the repelling of water molecules. The values highlighted in red do not provide useful information since the contact angle in between formulation is not of relevance. However, it gives useful information in terms of compatibility. All formulations are water based but using hydrophobic polymers. This means that they will be more compatible with the simulated coating than pure water, due to the presence in dispersion of hydrophobic compounds. Accordingly, pure water showed higher contact angle onto the simulated coating, than all the formulations. Regarding the contact angle onto a glass slide, as expected, water shows the lowest contact angle corresponding with the highest wettability degree. The closest to this value will mean the highest suitability for a film-forming ability, and the generation of a continuous coating with no porous. However, this value can be affected by the viscosity of the sample, increasing the contact angle due to a higher viscosity of the formulation.

FIG. 8. Data generated from UHT trials using microcapsules loaded with probiotics and exposed to UHT process of 140° C. for 3 seconds. Free bacteria are unable to survive this process.

FIG. 9. Image A illustrates UHT beverage background with evidence of high protein and fat content as per the staining protocol used. Image B and C identifies intact beads after UHT processing. Image D shows the protein and oil-stained microbeads.

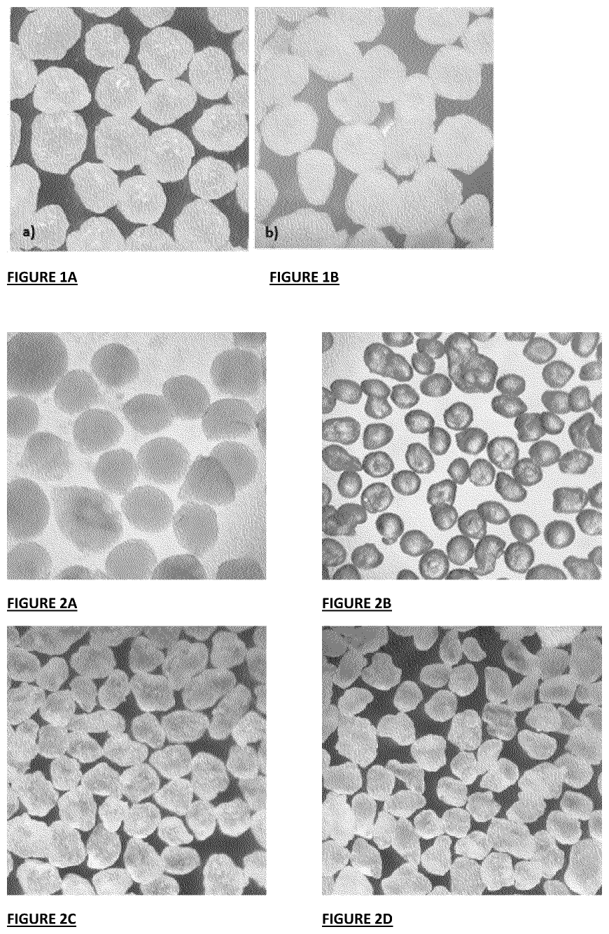

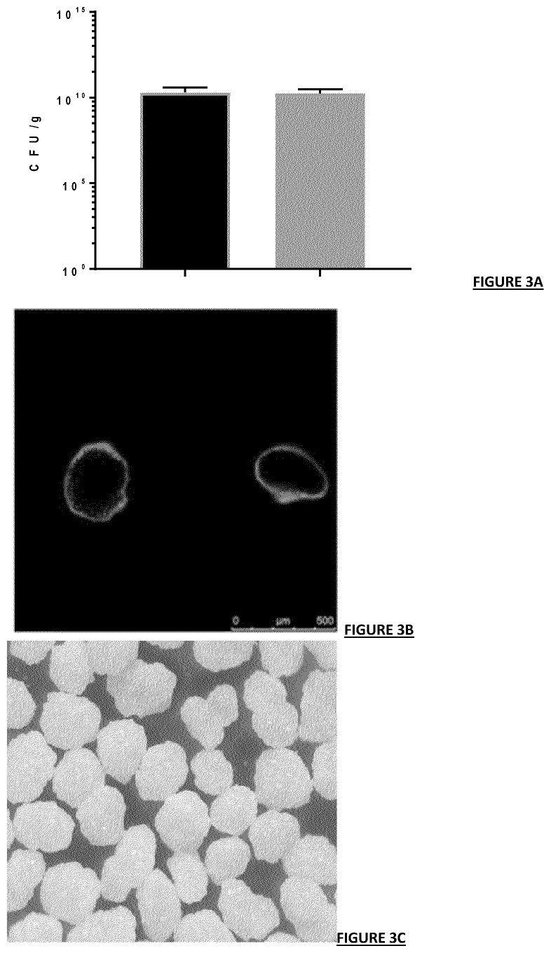

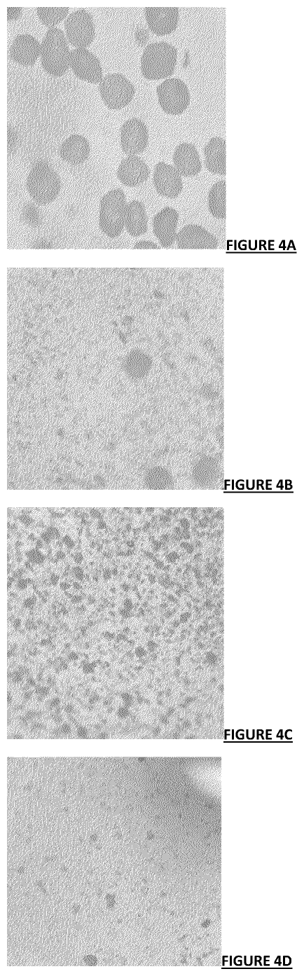

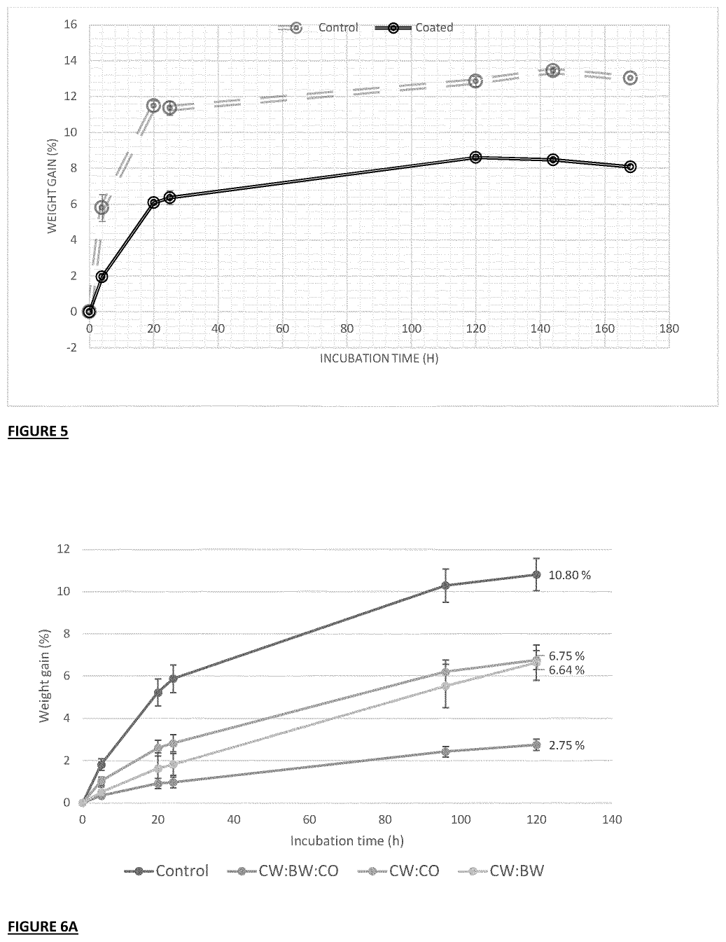

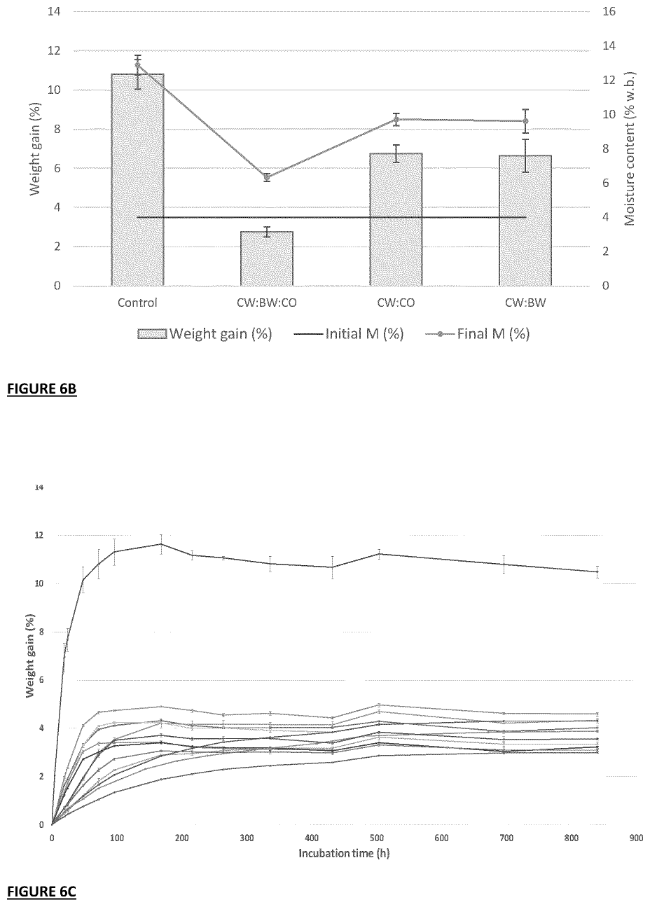

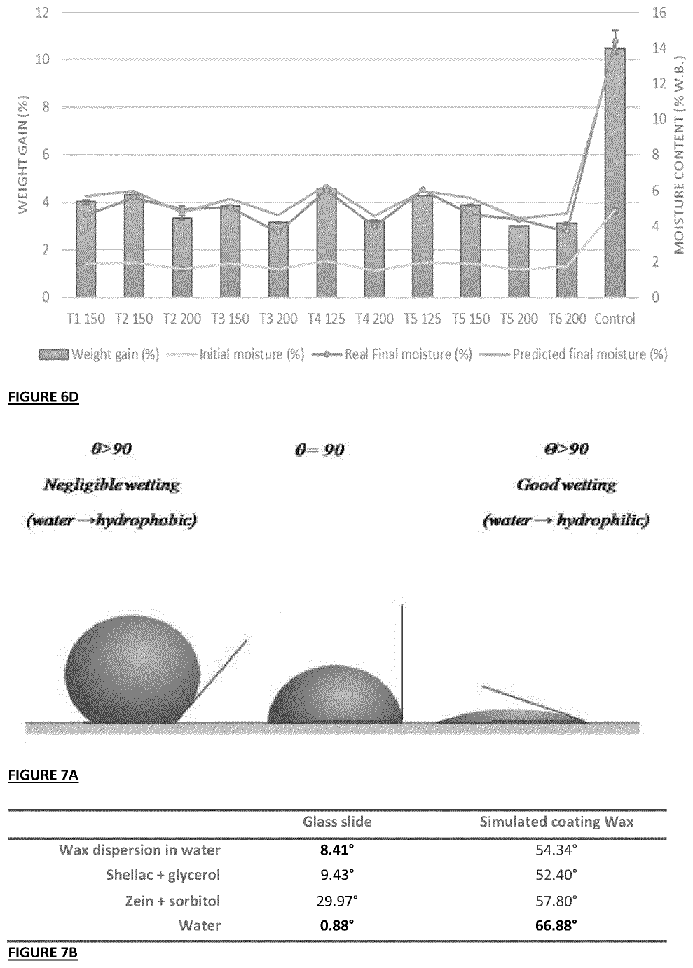

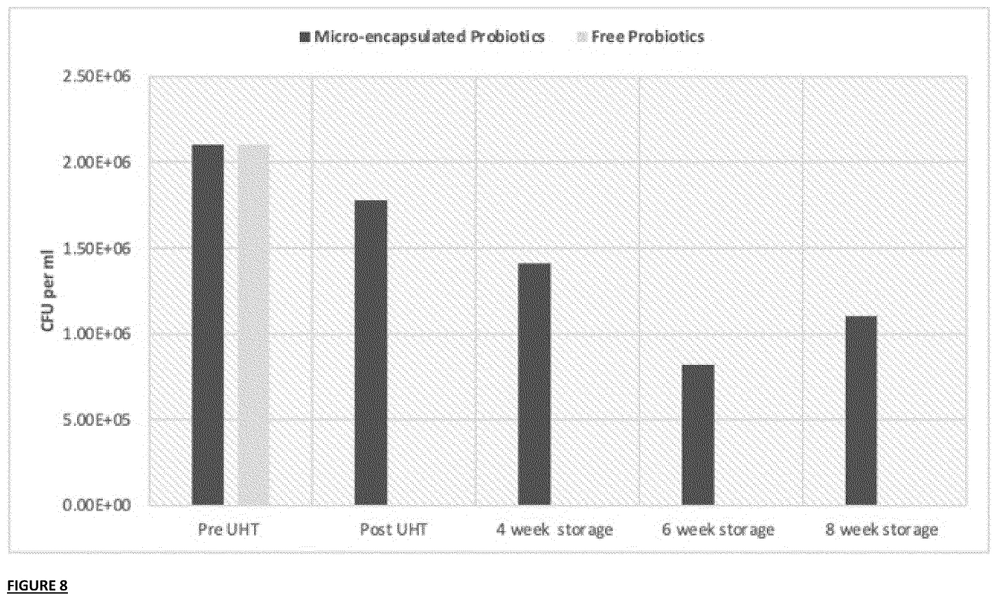

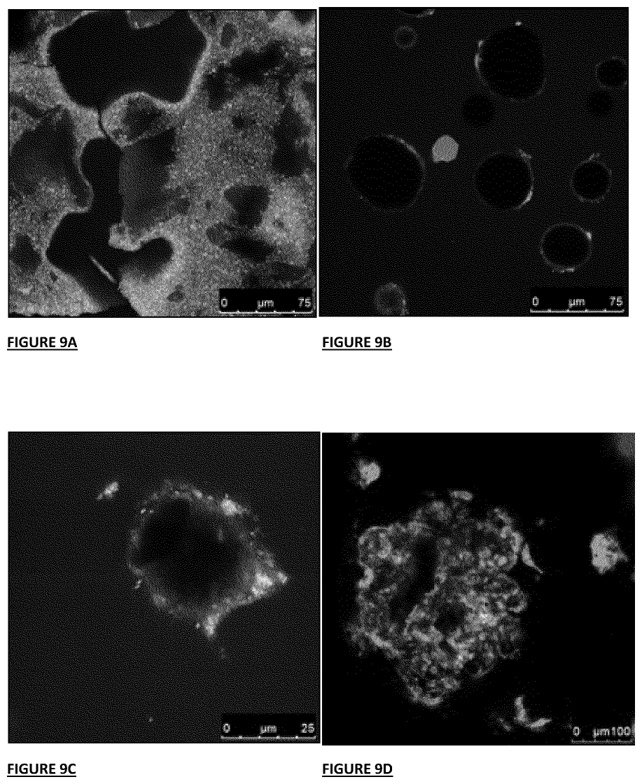

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and general preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

“Microcapsules” should be understood to mean generally spherical particles comprising gelled polymer for example denatured protein having an average diameter of 50 to 500 microns as determined using the light microscopy method described below. Preferably the microparticles have an average diameter of 50-200 microns as determined using the light microscopy method described below. Preferably the microparticles have an average diameter of 80-200 microns as determined using the light microscopy method described below. Preferably the microparticles have an average diameter of 80-150 microns as determined using the light microscopy method described below. Depending on the method of manufacture, the microparticles may be microbeads or microcapsules. The microcapsules may have a continuous polymerised matrix and an active agent dispersed throughout the pea protein matrix (i.e. multinuclear). Methods of producing multinuclear microcapsules are described in WO2016/096929. The microcapsules may have a core-shell morphology (i.e. mononuclear—active agent core and protein matrix shell). The core may be a liquid or a solid, or indeed a gas. Methods of producing mononuclear microcapsules are described in WO2014/198787.

“Meltable coating composition” refers to a composition comprising a wax component and an oil component that that is configured to be solid at ambient (i.e. room) temperature (i.e. at or below 25° C.) and have a melting point temperature that does not damage the matrix or active agent. Generally, the melting point temperature is less than 100° C. although for some protein matrices and active agents higher melting pointing temperatures may be employed. . Preferably, the coating composition has a melting point of 70-100° C., 80-100° C., 90-100° C., 65-100° C., 65-90° C., 70-90° C., 80-90° C., and ideally about 95° C. Typically, the coating composition is prepared my heating the wax and oil component to a melting temperature, and then ideally homogenising the wax and oil to provide the meltable coating composition. Generally, the melting point of the wax is different to the melting point of the oil

“Wax” refers to one or more natural waxes of plant or animal origin. Generally the wax has not been subjected to secondary chemical treatment such as hydrogenation. Examples of plant waxes include carnauba, berry, myrica fruit, candelilla, tea, rice bran, sunflower wax.

Examples of animal waxes include beeswax, Chinese, lanolin, shellac, and spermaceti waxes. Preferably, the wax is selected from beeswax and carnauba wax. Preferably the wax comprises a combination of beeswax and carnauba wax. In one embodiment the coating comprising comprises 40-90% wax, 50-80% wax, 55-75% wax, 60-70% wax, and ideally about 66% wax. In one embodiment, the coating comprises beeswax plus an additional wax, for example a plant wax such as carnauba wax. In one embodiment the coating comprising comprises 20-50%, 20-40%, 30-40%, or about 33% beeswax. In one embodiment, the beeswax makes up about 30-70% or 40-60% of the wax component of the coating comprising. In one embodiment, the wax has a melting point above 40° C., 50° C., 60° C., 70° C., 80° C. or 90° C.

In one embodiment, the coating composition comprises a wax component and an oil component. “Oil” refers to a plant or animal oil or butter, preferably a plant oil or butter. In one embodiment, the oil or butter is solid at room temperature and has a melting point above 20° C., 30° C., 40° C., 50° C., 60° C. or 70° C. Examples of oils or butter include coconut, cocoa, avocado, olive, stearic acid, sunflower, palm or sweet almond butters or derived oils, including chemical treatment such as hydrogenation.

The microcapsules are generally produced by cold gelation, which means extrusion of microdroplets, and immersion of the microdroplets in a gelation bath configured to cause the protein to polymerise to form a gelled bead. The gelling bath may be an acidic gelling bath.

“Extrusion” typically means passing the solution/suspension through a small orifice whereby the solution is broken up into micro-size droplets. Preferably, the solution is extruded through an orifice. Various methods will be apparent to the skilled person for generating droplets, for example prilling and spraying (ie spray drying). A preferred method of producing the microdroplets is a vibrating nozzle technique, in which the suspension is sprayed (extruded) through a nozzle and laminar break-up of the sprayed jet is induced by applying a sinusoidal frequency with defined amplitude to the spray from the nozzle.

Examples of vibrating nozzle machines are the Encapsulator and a machine produced by Nisco Engineering AG. Typically, the spray nozzle has an aperture of between 50 and 600 microns, preferably between 50 and 200 microns, suitably 50-200 microns, typically 50-150 microns, and ideally about 80-150 microns. Suitably, the frequency of operation of the vibrating nozzle is from 900 to 3000 Hz. Generally, the electrostatic potential between nozzle and acidification bath is 0.85 to 1.3 V. Suitably, the amplitude is from 4.7 kV to 7 kV. Typically, the falling distance (from the nozzle to the acidification bath) is less than 50 cm, preferably less than 40 cm, suitably between 20 and 40 cm, preferably between 25 and 35 cm, and ideally about 30 cm. The flow rate of suspension (passing through the nozzle) is typically from 3.0 to 10 ml/min; an ideal flow rate is dependent upon the nozzle size utilized within the process.

“Acidic gelling bath” means a bath having a pH below the pl of the matrix protein that is capable of instantaneously gelling the droplets. Typically, the acidic gelling bath has a pH of about 4.6. The acidic gelling bath is generally formed from an organic acid. Ideally, the acid is citric acid. Typically, the acidic gelling bath has an acid concentration of 0.1M to 1.0M, preferably 0.3M to 0.7M, and more preferably 0.4M to 0.6M. Typically, the acidic gelling bath has a citric acid concentration of 0.1M to 1.0M, preferably 0.3M to 0.7M, and more preferably 0.4M to 0.6M. Preferably, the acidic gelling bath comprises 0.4 to 0.6M citric acid and has a pH of less than 4.8, typically 4.4 to 4.7.

“Cured in the acidic gelling bath” means that the microbeads are allowed to remain in the gelling bath for a period of time sufficient to cure (harden) the microbeads. The period of time varies depending on the microbeads, but typically a curing time of at least 30 minutes is employed.

“Nozzle assembly” means an apparatus comprising at least one nozzle that is configured for extruding the protein solution through the at least one nozzle. In one embodiment, nozzle assembly comprises a single nozzle, whereby a mixture of active agent and protein solution may be extruded the single nozzle to form droplets which when gelled form microbeads. In another embodiment, the nozzle assembly comprises an outer nozzle concentrically arranged around an inner nozzle, and in which the protein solution is extruded through the outer nozzle and an active agent solution/suspension/dispersion is extruded through the inner nozzle to form droplets which when gelled form microencapsulates with a gelled protein shell and an active agent containing core. In another embodiment, the nozzle assembly comprises an outer nozzle concentrically arranged around an inner nozzle, and in which the protein solution and active agent is extruded through the inner nozzle and the coating compositions is extruded through the outer nozzle to form droplets which when gelled form microencapsulates with a gelled active agent containing core surrounded by a shell of coating composition.

“Active agent” means any component suitable for delivery to the mammalian small intestine or ileum, but typically means a component that is sensitive to an external condition for example heat, pH, pressure, chemical stress or enzymes. Thus, the active component may be sensitive to pH, enzymes (i.e. protease enzymes), high pressure, high shear, and temperature abuse during storage. In one particularly preferred embodiment of the invention, the active component is a cell, typically a bacterial cell, and ideally a probiotic cell. Such cells are sensitive to low pH conditions, such as would be encountered in the stomach, and as such need to be shielded from gastric pH and bile salt environments. Probiotic bacteria, and indeed other types of cells, are also sensitive to high shear or high pressure, such as are employed in conventional methods of generating micron-sized polymer beads. Other types of active components which may be encapsulated in the microbeads of the invention include micronutrients, vitamins, minerals, enzymes, starter bacteria, cell extracts, proteins and polypeptides (native or denatured), sugars and sugar derivatives, nucleic acids and nucleic acid constructs, pharmaceutically-active agents, imaging dyes and ligands, antibodies and antibody fragments, phytochemicals and the like.

“Active agent solution” means an active agent contained within a suitable liquid carrier in the form of a solution, dispersion or suspension.

“Drying” as applied to the microcapsules uses powder drying technology such as vacuum drying, fluidised bed drying or spray drying. Generally, the microcapsules are dried to a low moisture level, for example a water activity (a_{w}) of less than 0.4, 0.3 or 0.2. In one preferred embodiment, the microcapsules are dried using vacuum drying. Powder drying techniques are described in the following: Broeckx, G., Vandenheuvel, D., Claes, I. J., Lebeer, S., & Kiekens, F. (2016). Drying techniques of probiotic bacteria as an important step towards the development of novel pharmabiotics. International Journal of Pharmaceutics, 505(1-2), 303-318; Singh, S., & Dixit, D. (2014). A review on spray drying: Emerging technology in food industry. International Journal of Applied Engineering and Technology, 4(1), 1-8. Emami, F., Vatanara, A., Park, E., & Na, D. (2018). Drying technologies for the stability and bioavailability of biopharmaceuticals. Pharmaceutics, 10(3), 131. Benelli, L., Cortes-Rojas, D. F., Souza, C. R. F., & Oliveira, W. P. (2015). Fluid bed drying and agglomeration of phytopharmaceutical compositions. Powder technology, 273, 145-153.

“Water activity” (a_{w}) is the partial vapor pressure of water in a substance divided by the standard state partial vapor pressure of water. It is measured by the method described in Carter, B. P., Galloway, M. T., Campbell, G. S., & Carter, A. H. (2015). The critical water activity from dynamic dewpoint isotherms as an indicator of pre-mix powder stability. Journal of Food Measurement and Characterization, 9(4), 479-486. The operator's manual of the equipment used is provided at http://manuals.decaqon.com/Manuals/13893_AquaLab%20Pre_Web.pdf

“Coating the microcapsules” refers to the treatment of the microcapsules with the meltable coating composition resulting in the microcapsules being coating with a film of meltable coating composition. Methods of coating include mixing the coating composition with the dried microcapsules in a vessel. The meltable coating composition is heated to melt the coating composition, added to the microcapsules, and agitated until the microcapsules have been coated and the coating has solidified. Alternatively, the microcapsules can be coated with coating composition using a fluidised bed dryer (Teunou, E., & Poncelet, D. (2002). Batch and continuous fluid bed coating—review and state of the art. Journal of food engineering, 53(4), 325-340)). Generally, the coating step employs coating composition and microcapsules in a weight ratio of about 0.5:1.0 to 1.0:5, preferably 0.75:1.0 to 1.0:0.75, and ideally a ratio of about 1:1.

### EXEMPLIFICATION

The invention will now be described with reference to specific Examples. These are merely exemplary and for illustrative purposes only: They are not intended to be limiting in any way to the scope of the monopoly claimed or to the invention described.=These examples constitute the best mode currently contemplated for practicing the invention.

### Example 1

### Process of Making Coated Micro-Capsules

A probiotic frozen biomass was homogeneously dispersed in heat denatured protein solution (approximately 9-15% solid content).

This mixture is extruded throughout a vibrating nozzle (150-450 micron). Polymerisation is induced using an acidification bath comprised by the buffer of a sodium salt of an organic acid (0.1-1 M), and a divalent inorganic salt (0.1-1 M), adjusted to a final pH of 4.0-5.2.

The resultant wet microcapsules were collected and washed. The microcapsules were dried to moisture content of <4.0% and Aw of 0.2.

Separately, a coating solution consisting of a mixture of carnauba wax, beeswax and coconut oil (weight ratio 1:1:1) was prepared by heating to 95° C. and homogenised using a high sheer mixer at 10,000 rpm for 60 seconds.

The subsequent blend was maintained at 70-95° C. until required.

In a separate container, dry microcapsules and the coating solution were mixed in a ratio of 1:1 and agitated until the coating solution solidified due to a reduction in temperature yielding individual coated microcapsules.

Microcapsules were collected and sieved for and analysed for moisture content of <4.0% and Aw of 0.2.

### Example 2

### Process of making Coated Micro-Capsules

Dried microcapsules were produced according to Example 1.

Separately a coating solution consisting of a mixture of mixture of carnauba wax and coconut oil (weight ratio 1:1) was prepared by heating to 70-95° C. and homogenised using a high sheer mixer at 10,000 rpm for 60 seconds.

The subsequent blend was maintained at 95° C. until required.

In a separate container, dry microcapsules and the coating solution were mixed in a ratio of 1:1 and agitated until the coating solution solidified due to a reduction in temperature yielding individual coated microcapsules.

Microcapsules were collected and sieved for and analysed for moisture content of <4.0% and Aw of 0.2.

### Example 3

### Process of Making Coated Micro-Capsules

Dried microcapsules were produced according to Example 1.

Separately, a coating solution consisting of a mixture of carnauba wax, beeswax and coconut oil (weight ratio 1:1:1) was prepared by heating to 70-95° C. and homogenised using a high sheer mixer at 10,000 rpm for 60 seconds.

The subsequent blend was maintained at 95° C. until required.

Microcapsules were collected and sieved for and analysed for moisture content of <4.0% and Aw of 0.2.

### Example 4

### Process of Making Coated Micro-Capsules

Dried microcapsules were produced according to example 1.

Separately a coating solution consisting of a mixture of carnauba wax, beeswax and coconut oil (weight ratio 1:1:1) was prepared by heating to 70-95° C. and homogenised using a high sheer mixer at 10,000 rpm for 60 seconds.

The subsequent blend was maintained at 95° C. until required.

The dried microcapsules were subsequently fluidised using an appropriate apparatus keeping the coating solution at a temperature of 70-150° C.

The coating solution was sprayed utilising a heated vessel, tubing and nozzle setup (whereby the temperature of the blend was maintained as required) onto the fluidised microcapsules.

The amount of solution sprayed onto the capsules was between 20-200% of the original weight of the microcapsule powder in the fluidisation chamber.

### Example 5

### Conducting Stability on Coated Micro-Capsules

The hydrophobic properties imparted via the coating process were analysed by placing non-coated microcapsules (as described in example 1) and coated microcapsules (as described in example 3) in a high humidity environment (relative humidity 75%) for 120-800 hours).

Prior to the test, coated microcapsules were standardised in their superficial moisture at 10-15% RH for 24 h. Moisture uptake as a function of weight gain (%) with respect to the initial weight was recorded to demonstrate the hydrophobicity of the coating material. Non-coated microcapsules increased in weight by 10.8 to 11% while coated microcapsules increased in weight by approximately 2 to 6.8%, depending on the formulation.

The final moisture content (%) after 120-800 h of incubation at 71-75% RH for the control microcapsules and the coated microcapsules, was mathematically calculated from a normalized initial moisture content (%) of 4% w.b. using the weight gain (%) with respect to the initial weight of each formulation.

### Example 6

### Process of Making Coated Micro-Capsules

Disperse freezed dried probiotic culture in protein solution (10-15%) and agitate for 20 minutes at room temperature

Add denatured protein (9%-15% protein content) to the probiotic suspension and agitate.

Feed this probiotic-protein slurry to the inner core nozzle of a double concentric nozzle extruder

Feed a solution of prewarmed wax—oil solution to the outer shell nozzle of the double concentric nozzle extruder.

The ratio of each nozzle diameter can vary depending on the % coating needed

Maintain both premixes under slight stirring during the process (100 rpm).

Adjust the pressure to generate a stable bead chain for free fall into the polymerization buffer

Typically a pressure of 50-800 mBar is adequate for such probiotic—wax—oil suspensions

Microcapsules are polymerized in a citrate and /or acetate buffer (0.2-0.6M) at temperature between 15-59° C.

Collect the microcapsules generated and wash in sterile water.

Dry microcapsules to a moisture content less 4% and Aw less than 0.2.

The oil+wax coating layer applied can range from 20% to 200% of the original solids content of the feed.

### Example 7

### Process of Making Coated Micro-Capsules

Disperse probiotic culture in protein powder and agitate for 20 minutes at room temperature

Add denatured protein (9%-15% protein content) to the probiotic suspension and agitate.

Feed this probiotic-protein slurry to the inner core nozzle

Feed a solution of denatured protein (5%-12% protein content) to the outer shell nozzle.

The ratio of each nozzle diameter can vary depending on the % coating needed

Maintain both premixes under slight stirring during the process (100 rpm).

Adjust the pressure to generate a stable bead chain for free fall into the polymerization buffer

Typically a pressure of 50-800 mBar is adequate for such probiotic—protein suspensions

Microcapsules are polymerized in a citrate and/or acetate buffer (0.2.-0.6M) at temperature between 15-50° C.

Collect the microcapsules generated and wash in sterile water.

Dry microcapsules to a moisture content less 4% and Aw less than 0.2.

The protein coating layer applied can range from 20% to 200% of the original solids content of the feed.

### Example 8

### Process of Making Protected Microparticles

Disperse freeze dried probiotic culture in a protein powder (with min. calcium content of 1%) powder

Mix powder for 20 minutes in dry blender

Fluidise the powder blend for 20 minutes at 40° C. fluidised using an appropriate apparatus

Coat the fluidised powder with a denature protein solution at a temperature of 20-40° C.

The coating solution is sprayed using a nozzle diameter from 50-600 um

The amount of solution sprayed onto the protein and culture is between 10-200% of the original weight of the protein powder

Collected microparticles and sieve.

Dry microparticles should have a moisture content less 4% and Aw less than 0.2.

### Equivalents

The foregoing description details presently preferred embodiments of the present invention.

Numerous modifications and variations in practice thereof are expected to occur to those skilled in the art upon consideration of these descriptions. Those modifications and variations are intended to be encompassed within the claims appended hereto.

1. A method of producing coated microcapsules comprising the steps of:
producing cold-gelated microcapsules having an average diameter of 50-500 microns and a denatured or hydrolysed protein matrix and an active agent contained within the matrix;
drying the microcapsules;
providing a meltable coating composition comprising wax and oil having a melting point of 70-100° C.;
heating the meltable coating composition to a temperature above the melting point of the meltable coating composition to melt the meltable coating composition; and
coating the microcapsules with the melted meltable coating composition.

2. A method according to claim 1, in which the coating composition comprises 50-90% wax and 10-50% oil.

3. A method according to claim 1 or 2, in which the coating composition comprises 60-70% wax and 30-40% oil.

4. A method according to any preceding Claim, in which the protein is denatured protein.

5. A method according to any preceding Claim in which the matrix consists essentially of polymerised protein.

6. A method according to any preceding Claim, in which the wax comprises carnauba wax.

7. A method according to any preceding Claim, in which the oil comprises coconut oil.

8. A method according to any preceding Claim in which the coating composition comprises carnauba wax and beeswax or coconut oil.

9. A method according to any preceding Claim in which the coating composition comprises carnauba wax, beeswax, and coconut oil.

10. A method according to any preceding Claim, in which the drying step comprises drying the microcapsules to water activity a_{w} of less than 0.3 at 25° C.

11. A method according to any preceding Claim, in which the drying step comprises drying the microcapsules to water activity a_{w} of less than 0.2 at 25° C.

12. A method according to any preceding Claim, in which the drying step comprises a primary drying stage and a secondary drying stage in a low humidity environment.

13. A method according to any preceding Claim, in which the coating step comprises mixing the heated coating composition and dried microcapsules in a coating chamber until the coating composition has coated the microcapsules and solidified.

14. A method according to any of claims 1 to 12, in which the coating step comprises spraying the heated coating comprising on to the dried microcapsules on a fluidised bed.

15. A method according to claim 13 or 14, in which the heated coating composition is homogenised at high shear prior to coating the microcapsules.

16. A method according to any preceding Claim, in which the coating step employs about 40-60% coating composition and about 40-60% dried microcapsules.

17. A method according to any preceding Claim, in which the matrix comprises denatured vegetable or milk protein.

18. A method according to any preceding Claim, in which the active agent comprises a probiotic bacteria, oil, or micronutrient composition.

19. A method according to any preceding Claim, in which the microcapsules are produced by extruding microdroplets comprising denatured or hydrolysed protein and an active agent into a gelation bath, and gelation of the microdroplets in the gelation bath to provide microcapsules.

20. A method according to claim 19, in which the microcapsules are multinuclear, in which the microcapsules are produced by providing a suspension comprising denatured or hydrolysed protein matrix and an active agent, and extruding microdroplets of the suspension into a gelation bath.

21. A method according to any of claims 1 to 18, in which the microcapsules are mononuclear, in which the microcapsules are produced by providing a first liquid composition comprising denatured or hydrolysed protein matrix and a second liquid composition comprising an active agent, and co-extruding the first and second liquid compositions using concentric nozzles in which the first liquid composition is extruded through an outer nozzle and the second liquid composition is extruded through an inner nozzle to form core-shell microdroplets, and gelation of the core-shell microdroplets in a gelation bath.

22. A method according to any of claims 1 to 18, in which the microcapsules are produced by providing a first liquid composition comprising denatured or hydrolysed protein matrix and an active agent, and co-extruding the first liquid compositions and the meltable coating composition using concentric nozzles in which the first liquid composition is extruded through an inner nozzle and the second liquid composition is extruded through an outer nozzle to form core-shell microdroplets in which the shell comprises the meltable coating composition, and gelation of the core-shell microdroplets in a gelation bath.

23. A microcapsule comprising:
a crosslinked denatured or hydrolysed protein matrix;
an active agent contained within the matrix;
wherein the microcapsule is coated with a coating composition comprising wax and oil and having a melting point of 70-100° C.

24. A microcapsule according to claim 23, in which the coating composition comprises 50-90% wax and 10-50% oil.

25. A microcapsule according to claim 24, in which the coating composition comprises 60-70% wax and 30-40% oil.

26. A microcapsule according to any of claims 23 to 25, in which the protein is denatured protein.

27. A microcapsule according to any of claims 23 to 26, in which the wax comprises carnauba wax.

28. A microcapsule according to any of claims 23 to 27, in which the wax comprises beeswax and/or carnauba wax.

29. A microcapsule according to any of claims 23 to 28, in which the oil comprises coconut oil.

30. A microcapsule according to any of claims 23 to 29, in which the coating composition comprises carnauba wax and beeswax or coconut oil.

31. A microcapsule according to any of claims 23 to 29, in which the wax comprises carnauba wax, beeswax, and coconut oil.

32. A microcapsule according to any of claims 23 to 31, in which the microcapsule has a water activity Aw of less than 0.3 at 25° C.

33. A microcapsule according to any of claims 23 to 32, in which the microcapsule has a water activity Aw of less than 0.2 at 25° C.

34. A microcapsule according to any of claims 23 to 33, in which the microcapsule is a mononuclear microcapsule.

35. A microcapsule according to any of claims 23 to 33, in which the microcapsule is a multinuclear microcapsule.

36. A microcapsule according to any of claims 23 to 35, in which the protein is selected from a milk or vegetable protein, which is denatured.

37. A heat-treated composition comprising microcapsules according to any of claims 23 to 36, in which the composition is heat treated at a temperature less than the melting point of the coating composition.

38. A heat-treated composition according to claim 37 which is pasteurised.

39. A method of making a heat-treated composition comprising the steps of providing a composition comprising microcapsules according to any of claims 23 to 36, and heat treating the composition at a temperature less than the melting point of the coating composition.

40. A method according to claim 39, in which the heat treatment is pasteurisation.

41. A heat treated composition according to claim 37 or 38, or a method according to claim 39 or 40, in which the active agent comprises probiotic bacteria.
